(19) United States
(12) Patent Application Publication
(10) Pub. No.: US 2016/0331273 A1
(43) Pub. Date: Nov. 17, 2016

(54) METHOD AND APPARATUS FOR PROCESSING CARDIAC SIGNALS AND DERIVING NON-CARDIAC PHYSIOLOGICAL INFORMATOIN

(71) Applicant: THE GENERAL HOSPITAL CORPORATION, Boston, MA (US)

(72) Inventor: Antonis A. Armoundas, Boston, MA (US)

(21) Appl. No.: 15/111,638

(22) PCT Filed: Jan. 12, 2015
(86) PCT No.: PCT/US15/10959

§ 371 Date: Jul. 14, 2016

Related U.S. Application Data
(60) Provisional application No. 61/928,498, filed on Jan. 17, 2014.

(51) Int. Cl. A61B 5/08; A61B 5/04; A61B 5/0452; A61B 5/00; A61B 5/091; A61B 5/0408; A61B 5/0472

(52) U.S. Cl. CPC A61B 5/0816; A61B 5/04085; A61B 5/04012; A61B 5/0472; A61B 5/7257; A61B 5/091; A61B 5/04525

(57) ABSTRACT

A system and a method are provided for deriving elctrocardiographic (ECG] signals from a subject. The system includes an ECG apparatus configured to acquire ECG signals from the subject through a plurality of ECG leads, wherein the plurality of ECG leads includes lead groups that are traditionally presumed to be orthogonal. A processor or method are provided to analyze combinations of ECG leads from the plurality of ECG leads to determine a spectral signal-to-noise ratio (SNR] for each combination of ECG leads and select a combination of ECG leads that provides a desirable spectral SNR. The ECG signals derived from the combination of ECG leads selected as providing the desirable spectral SNR may be provided or may be used to derive and report respiratory rate information about the subject.

### CROSS-REFERENCE TO RELATED APPLICATIONS

The present invention is based, claims priority to, and incorporates herein by reference in its entirety, U.S. Provisional Application Ser. No. 61/928,498, filed Jan. 17, 2014, and entitled “METHOD AND APPARATUS FOR PROCSSSING CARDIAC SIGNALS AND DERIVING NON-CARDIAC PHYSIOLOGICAL INFORMATION.”

### STATEMENT REGARDING FEDERALLY SPONSORED RESEARCH

N/A

### BACKGROUND

The present disclosure is related to subject monitoring. More particularly, the disclosure relates to a method for determining a respiratory information, such as respiratory rate (RR), from intra-cardiac or body surface electrocardiographic signals.

Measurement of respiratory rate is a valuable component of patient monitoring and disease management in a number of clinical settings including ambulatory care, emergency rooms, post-operative care and intensive care units. For patients in a hospital setting, measurement of RR can be accomplished either directly or indirectly, using a number of different methods. Nasal thermocouples and spirometers directly measure air flow into and out of the lungs. Pulse oximetry, transthoracic inductance, impedance plethysmographs, pneumatic respiration transducers, and whole-body plethysmographs indirectly monitor RR by measuring body volume changes.

Common to all of these methods is the use of specialized hardware that is dedicated to RR monitoring, which is a feature that is not often practical and convenient in an emergency setting or for the free-moving, ambulatory patient. Assessment of the RR is important in the ambulatory monitoring of many diseases, including chronic obstructive pulmonary disease, sleep apnea, sudden infant death syndrome, and Cheyne-Stokes respiration (CSR) in heart failure. In particular, CSR is a form of sleep disordered breathing in which crescendo-decrescendo alterations in tidal volume are separated by periods of apnea and hypopnea. Cheyne-Stokes respiration has been identified in up to 40 percent of patients with chronic heart failure and has been associated with cardiac dysrhythmias including atrio-ventricular block and ventricular ectopy. Additionally, CSR is a marker of other prognosis and increased mortality in patients with heart failure and improvements in CSR might serve as a positive marker of response to heart failure medical therapy. These clinical observations exemplify the complex interplay between the respiratory, cardiovascular and autonomic systems and highlight the need for tools to monitor respiratory and cardiovascular parameters in ambulatory patients with heart failure.

Some have attempted to estimate the RR by extracting parameters of the respiratory signal from ECG signals. These efforts utilized signal processing techniques to assess the impact of changes in air-flow or body-volume on the ECG signal and estimate the respiratory rate. Such approaches are touted as being highly desirable in situations when the respiratory activity is impractical to monitor but the ECG is recorded, e.g., during a 24 -hour Holter ambulatory recording. However, some studies have reported a 6 percent error in the estimated versus measured RR, using spirometery as a gold standard or an average correlation of 80 percent between the estimated respiration signal and a chest-belt respiration sensor. An additional limitation of these methods is that they require a priori selection of the ECG leads to be used for estimation of RR and these selections cannot change once the estimation has started. This issue becomes especially problematic in the case of an implantable device (i.e. pacemaker or defibrillator) where the intra-cardiac electrograms (EGMs) could be used to estimate RR but it is often unclear which EGM configurations will provide the most accurate estimation of the RR.

Therefore, given these shortcomings, it would be desirable to have a system and method that facilitates the determination of respiratory rate of a subject without the need for specialized respiration monitoring systems and without requiring specialized or cumbersome configuration or setup of other monitoring systems to be suitable for deriving respiration information from the feedback of the other monitoring system.

### SUMMARY

The present disclosure overcomes the aforementioned drawbacks by providing a system and method for determining respiratory information about a subject from elctrocardiography (ECG) signals without requiring that the setup or configuration of the ECG monitoring system comply with some predetermined arrangement. For example, the present disclosure provides a system and method for determining respiratory information about a subject from ECG signals that are derived by ECG electrodes that may or may not be configured in a predetermined or preferred arrangement, such as in an orthogonal relationship.

In accordance with one aspect of the disclosure, a method is disclosed for determining the respiratory rate of a subject from elctrocardiographic (ECG) signals acquired from the subject. The method includes acquiring ECG signals from an ECG monitor coupled to the subject through a plurality of ECG leads, wherein the plurality of ECG leads includes traditionally-orthogonal lead groups that are non-orthogonal. The method also includes determining a combination of ECG leads from the plurality of ECG leads that provides a spectral signal-to-noise ratio (SNR) above a threshold value and processing the ECG signals from the determined combination of ECG leads to extract a respiratory rate of the subject from the ECG signals. The method further includes generating a report indicating a respiratory rate of the subject determined based on extracted reparatory rate.

In accordance with another aspect of the disclosure, a system is disclosed for determining a respiratory rate of a subject from elctrocardiographic (ECG) signals acquired from the subject. The system includes an ECG apparatus configured to acquire ECG signals from the subject through a plurality of ECG leads, wherein the plurality of ECG leads includes lead groups that are traditionally presumed to be orthogonal. The system also includes a processor configured to determine a combination of ECG leads from the plurality of ECG leads that provides a spectral signal-to-noise ratio (SNR) above a threshold value. The processor is further configured to process the ECG signals from the determined combination of ECG leads using an algorithm configured to extract a respiratory rate of the subject from the ECG signals. The system also includes a report generator configured to provide a report of the respiratory rate of the subject.

In accordance with another aspect of the disclosure, a system is disclosed for deriving elctrocardiographic (ECG) signals from a subject. The system includes an ECG apparatus configured to acquire ECG signals from the subject through a plurality of ECG leads, wherein the plurality of ECG leads includes lead groups that are traditionally presumed to be orthogonal. The system also includes a processor configured to analyze combinations of ECG leads from the plurality of ECG leads to determine a spectral signal-to-noise ratio (SNR) for each combination of ECG leads and, based thereon, select a combination of ECG leads that provides a desirable spectral SNR. The system further includes a report generator configured to provide a report of the ECG signals derived from the combination of ECG leads selected in by the processor as providing the desirable spectral SNR.

In accordance with another aspect of the disclosure, a method is disclosed for determining respiratory rate of a subject from elctrocardiographic (ECG) signals acquired from the subject. The method includes acquiring ECG signals from an ECG monitor coupled to the subject through a plurality of ECG leads, wherein the plurality of ECG leads includes lead groups that are presumed to be orthogonal. The method also includes analyzing combinations of ECG leads from the plurality of ECG leads, including lead groups other than the lead groups that are presumed to be orthogonal, to determine a combination of ECG leads that provides a spectral signal-to-noise ratio (SNR) greater that other combinations of ECG leads from the plurality of ECG leads. The method further includes tracking a dominant spectral peak in the ECG signals from the determined combination of ECG leads, correlating the dominant spectral peak with a respiratory rate of the subject, and generating a report indicating the respiratory rate of the subject based on the correlated respiratory rate.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is illustration of an elctrocardiography (ECG) monitoring system configured in accordance with the present disclosure.

FIG. 1B is a schematic illustration of a system such as described in the present disclosure for use with a mobile device.

FIG. 2 is a schematic diagram showing a standard, 12-lead ECG configuration for use in accordance with the present disclosure

FIG. 3 is a flowchart setting forth steps of an exemplary operation of the illustrative ECG monitoring system of FIG. 1A in accordance with the present disclosure.

FIG. 4 is a flowchart setting forth steps of an exemplary process for selecting a desired or optimal ECG lead combination in accordance with the present disclosure.

FIG. 5A shows a graphical example illustrating heart rate and respiration rate time recordings for optimal beat window selection.

FIG. 5B shows a graphical example illustrating theoretical estimations for transition time for a number of beat windows to reach a new rate, in accordance with the present disclosure.

FIG. 5C shows a graphical example illustrating average (across leads) respiratory rate plotted as a function of time, in accordance with the present disclosure.

FIG. 5D shows a graphical example illustrating standard deviation of respiratory rate estimates of FIG. 5A as a function of window length, in accordance with the present disclosure.

FIG. 6A shows a graphical example illustrating ventilator rate step-down adjustment.

FIG. 6B shows a graphical example illustrating normalized power spectrum as a function of time during a step down transition of the ventilation rate.

FIG. 7A shows a graphical example illustrating absolute error for unipolar leads.

FIG. 7B shows a graphical example illustrating absolute error for far-field bipolar leads.

FIG. 7C shows a graphical example illustrating absolute error for near-field bipolar leads.

FIC 7D shows a graphical example illustrating absolute error for RV-CS leads.

FIG. 8A shows a graphical example illustrating a percentage of missed detections for unipolar leads.

FIG. 8B shows a graphical example illustrating a percentage of missed detections for far-field bipolar leads.

FIG. 8C shows a graphical example illustrating a percentage of missed detections for near-field bipolar leads.

FIG. 8D shows a graphical example illustrating a percentage of missed detections for RV-CS leads.

FIG. 9A shows a graphical example illustrating signal to noise ratio (SNR) estimates for different intra-cardiac lead type, compiled across all animals, at tidal volumes, and ventilation rates, for all accurate and missed detections.

FIG. 9B shows a graphical example illustrating absolute error for lead groupings using an optimized algorithm in accordance with the present disclosure.

FIG. 9C shows a graphical example illustrating a percent of missed detections for each lead group, in accordance with the present disclosure.

FIGS. 10A-D show graphical examples illustrating estimated versus true respiration rates for several lead groupings using an optimized algorithm, in accordance with the present disclosure.

FIGS. 11A-C show graphical examples illustrating intra-cardiac only respiratory rate estimation, in accordance with the present disclosure.

FIGS. 12A-C show graphical examples illustrating tidal volume, respiratory rage estimation, and percent of optimized estimations, in accordance with the present disclosure.

### DETAILED DESCRIPTION

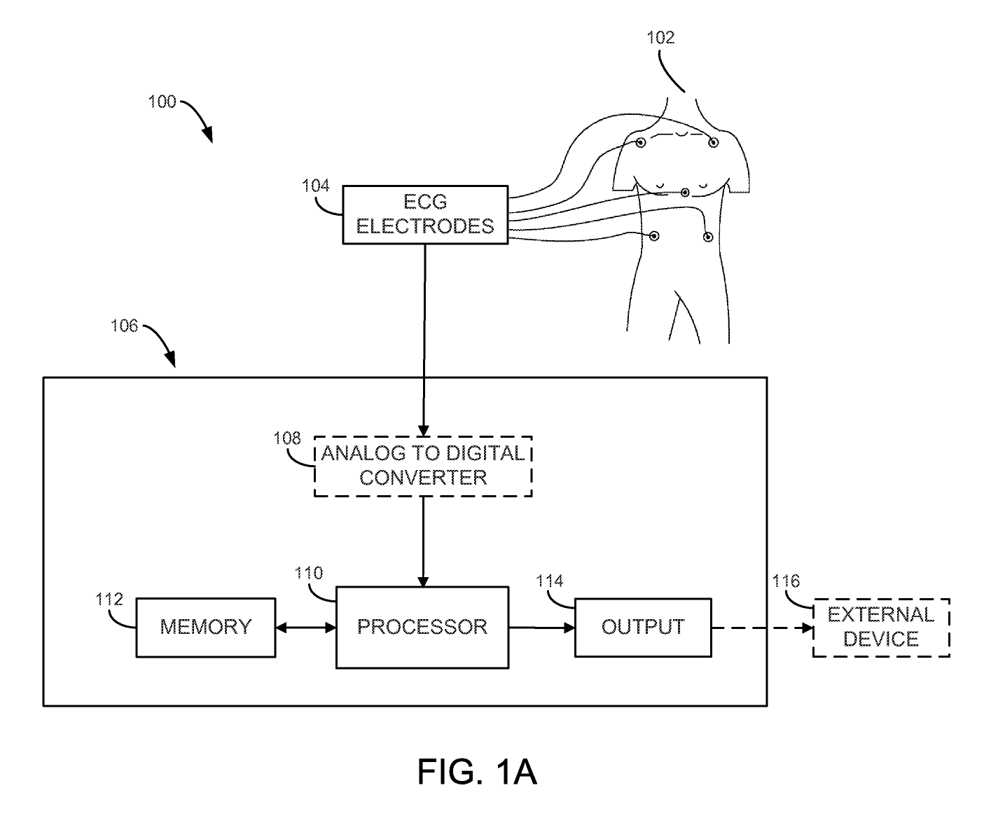

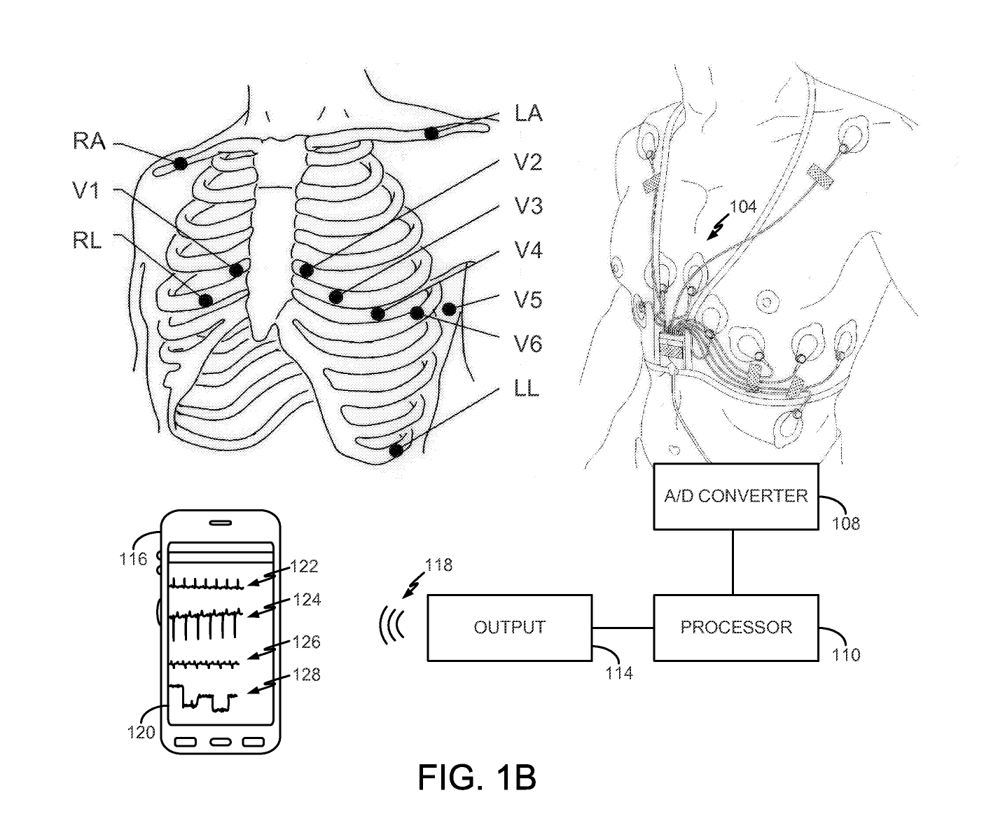

Turning to FIGS. 1A and 1B, an example for an ECG monitoring system 100 is shown, which may be any device, apparatus or system, or may operate as part of, or in collaboration with a computer, system, device, machine, or mainframe, server, or may be a mobile, a wearable device (e.g., bracelet or watch), or portable device. For example, the ECG monitoring system 100 may be a computer, mobile phone, tablet, or other personal electronic device. In this regard, the ECG monitoring system 100 may be a general computing device that may integrate a variety of software and hardware capabilities and functionality. In this regard, the ECG monitoring system 100 may transmit the recorded ECG signals using wireless communication, such as using Bluetooth or other communications protocols, to a mobile phone, tablet, or other personal electronic device or through a mobile phone, tablet, or other personal electronic device to an external device for further processing and estimation of the respiratory rate. The ECG monitoring system 100 includes a plurality of ECG electrodes 104 that may be disposed upon a surface of, or within, the anatomy of a subject 102 according to a desired configuration. That is, the ECG monitoring system 100 may be designed to operate with surface electrodes or implantable electrodes, including those associated with pacemakers or defibrillators. In this regard, “ECG data ” or “ECG signals” data may include data acquired from such surface or implanted or implantable electrodes and, thus, also includes intra-cardiac electrogram (EGM) data or EGM signals.

Such ECG signals from the ECG electrodes 104 are monitored and analyzed continuously or intermittently via an ECG monitoring apparatus 106. The ECG monitoring apparatus 106 may be configured to convert analog ECG signals to digital ECG signals and, thus, include an analog to digital converter 108. The ECG signals are communicated to a processor 110 and may be stored within and retrieved from a memory 112 or from an external device (i.e. cloud) for analysis and/or communicated to an output 114. The output 114 may be a display or printing system configured to generate a report.

Also, as illustrated in FIG. 1B, the output may be a communications output, for example, that is configured to communicate information via wired or wireless signals 118 to an external device 120. As illustrated in FIG. 1B, the external device 120 may be a mobile computing system, including a smartphone, a wearable device (e.g., a bracelet or watch), or tablet. In the illustrated, non-limiting example, a 12 lead ECG acquisition, display, and analysis system that formed by a 12-channel ECG module 104 (such as a PSL-ECG 12MD form Physiolab) an AD converter 108 (such as an ADS1298 from Texas Instruments) a microcontroller or processor 110 (such as a Due from Arduino), an output 114 (such as a Bluetooth communications UART converter, such as a HC-05 from Guangzhou HC Information Technology Co., Ltd), and a mobile device (such as a smartphone) 116. 10 ECG electrodes (RL, LL, RA, LA, V1-V6) are illustrated as connected to the ECG module 104. The A/D converter 108 amplifies and digitizes 8 leads (I, II, and V1-V6), for example, simultaneously at 500 samples/sec (SPS). Wilson's central terminal ((LA+RA+LL)/3) may be used as a reference potential for the precordial leads. The processor 110 can communicate with the A/D converter 108 via, for example, a wired communications link or connection and coordinate communication of acquired data to the mobile device 116 via the output 114, such as using wireless communications protocols. For example, the digitized 24 bit resolution signals may be transferred to the processor 110, which reduces the resolution to 16 bits in order to reduce the number of errors during wireless data transmission to the mobile device 116. The data received by the mobile device may then be displayed trough a display and associated user interface 120. As such, the mobile device 116 may calculate the 12 lead ECG signals (I, II, III, aVR, aVL, aVF, and V1-V6) from the 8 leads. As illustrated, the user may select to display multiple leads at any given time 122-126 and/or may view the respiration rate 128.

As will be described, the processor 110 may be further configured to determine respiratory information in accordance with the present disclosure about the subject 102 from the ECG signals. In some configurations, the processor 110 may also be capable of determining a tidal volume and estimating a minute ventilation using acquired ECG signals. For example, changes on a beat-by-beat basis of root-mean-square (RMS) amplitudes of the ECG signals may be used to compute a modulation of a respiration envelope signal. Such information may then be used by the processor 110 to identify an optimal lead configuration for a tidal volume analysis.

In this regard, the respiratory information, as well as any other information, may be communicated by the processor 110 through the output 114. Alternatively, the output 114 may be a data output configured to communicate the acquired ECG signals to an external device 116, which may function as a processing device to perform operations in accordance with the present disclosure and, thereby, determine and communicate respiratory information.

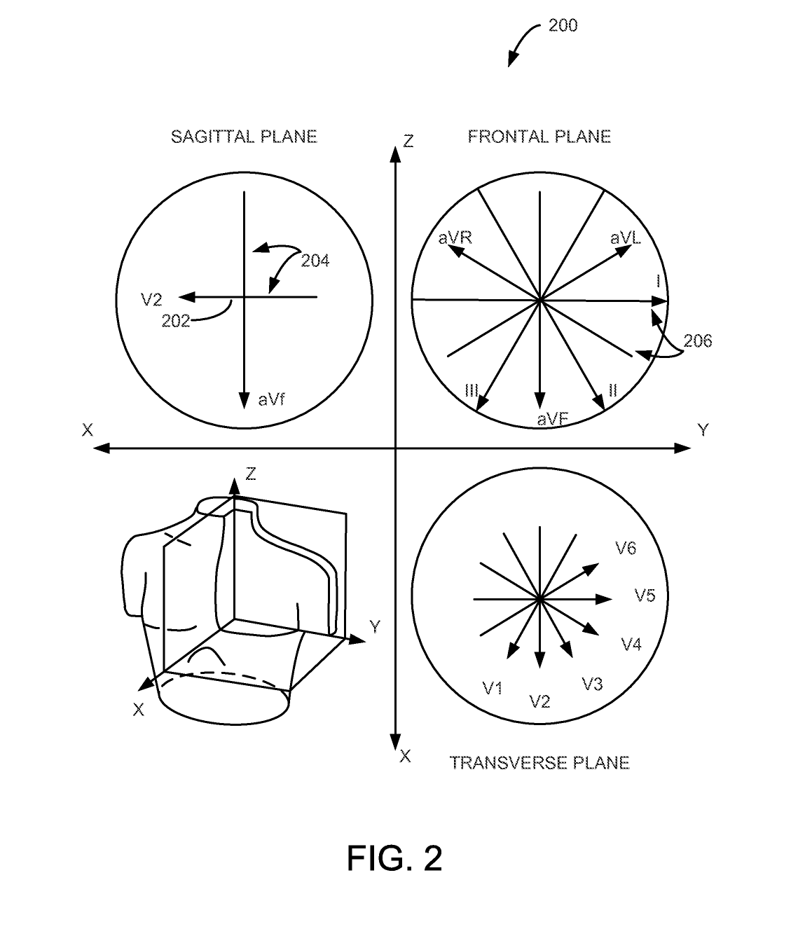

As is well-known in the art, an ECG lead may typically refer to the tracing of the voltage difference between two ECG electrodes, wherein the naming of an ECG lead in a particular configuration makes reference to the electrical polarity and placement location of the ECG electrodes. Signals from ECG leads may be obtained from explicit measurement of voltage difference between two physical ECG electrodes, known in the art as bipolar ECG leads, or measurement of voltage differences between a single physical ECG electrodes and combinations of signals from other ECG electrodes. Referring to FIG. 2, a 12-lead ECG configuration 200 is illustrated, which is a configuration common in clinical use. A given direction along an ECG lead 202 is known in the art as a lead axis. As shown in FIG. 2, lead axes may be orthogonal 204 (i.e. oriented substantially perpendicular to one another) and other lead axes may be non-orthogonal 206.

Most ECG monitoring system and methods require and/or assume that particular combinations of ECG leads will be arranged orthogonally because an orthogonal relationship between combinations of leads provides optimal signal strength, typically calculated as a signal-to-noise ratio (SNR). As such, traditional ECG systems require operators or clinicians to specifically configure combinations of ECG leads to be arranged orthogonally. For example, many ECG monitors expect a SNR achievable only with substantial (i.e., within a few degrees) orthogonality or such ECG monitors may base calculations upon a specific assumption of orthogonality. For example, when the leads are orthogonal, the arctangent of the ratio of the QRS areas measured in the two leads results in the angle (theta) of the mean axis with respect to one of the lead axes. A lack of orthogonality results in diminished results or inaccurate calculations.

In particular, with reference to FIG. 2, orientation of a 12-lead ECG system typically provides spatial information about the heart's electrical activity in three orthogonal directions: left/right, superior/inferior, and anterior/posterior. Each of the 12 leads represents a particular orientation in space, as indicated below (RA=right arm; LA=left arm, LL=left foot). Bipolar limb leads (frontal plane) include Lead I-RA (−) to LA (+) (Right Left, or lateral); Lead II-RA (−) to LL (+) (Superior Inferior); and Lead III-LA (−) to LL (+) (Superior Inferior). Augmented bipolar limb leads (frontal plane) include Lead aVR-RA (+) to [LA & LL] (−) (Rightward); Lead aVL-LA (+) to [RA & LL] (−) (Leftward); and Lead aVF-LL (+) to [RA & LA] (−) (Inferior). Finally, bipolar chest leads (horizontal plane) include Leads V1, V2, V3: (Posterior Anterior) and Leads V4, V5, V6:(Right Left, or lateral). Thus, within each of these various and common ECG lead configurations, there are various lead combinations that represent lead combinations presumed to be orthogonal. A failure to maintain the requisite orthogonality of these lead combinations presumed to be orthogonal or between traditionally-orthogonal groups or pairs of leads that are dictated and assumed to be orthogonal in a given lead configuration is considered unfavorable for the reasons explained.

However, the present disclosure provides a system and method to determine a combination of ECG leads from the plurality of ECG leads that provides a desired or optimal SNR above a threshold value and process the ECG signals from the combination of ECG leads determined using an algorithm configured to extract a respiratory rate of the subject from the ECG signals. Based on the determined SNR, the present disclosure can compensate for or calibrate for non-orthogonality and, using the information provided by such lead combinations, provide an ECG-derived respiration measurement surrogate. In this regard, the present disclosure removes the need to predefine a lead configuration and, within a predefined lead configuration known to include lead combinations presumed to be orthogonal, allows such traditionally-orthogonal groups or pairs of leads to be non-orthogonal. That is, the present disclosure can calibrate for, compensate for, or provide accurate feedback despite the presence of non-orthogonality of traditionally-orthogonal groups or pairs of leads.

Furthermore, some have determined that an ECG-derived respiration can be derived by using an estimation of the mean cardiac axis on a beat-by-beat basis, and deriving a respiration rate (RR) from this signal as the mean cardiac axis changes throughout the respiratory cycle. Specifically, as mentioned above, the angle of the mean cardiac axis with respect to one of the lead axes may be estimated by calculating the arctangent of the ratio of QRS amplitudes from two ECG leads. This respirophasic modulation is independent of electrode motion artifact or other sources of non-specific noise. The respiration frequency can then be estimated from the respirophasic signal using a spectral analysis method.

The present disclosure recognizes that it is often impractical to select orthogonal intracardiac leads, both because the identification of orthogonal ECG leads is very difficult, even under fluoroscopy, and because lead motion may cause the angle between two leads to change as a function of respiration or posture. In addition, not only the mean cardiac axis but also the thoracic impedance changes as a function of respiration, such that the angle of the mean cardiac axis is not perfectly described by the arctangent of the ratios of orthogonal leads. Therefore, the current disclosure describes an approach that can accurately and reliably estimate the respiration rate from non-orthogonal ECG lead combination, and without calculating the arctangent of the QRS ratios.

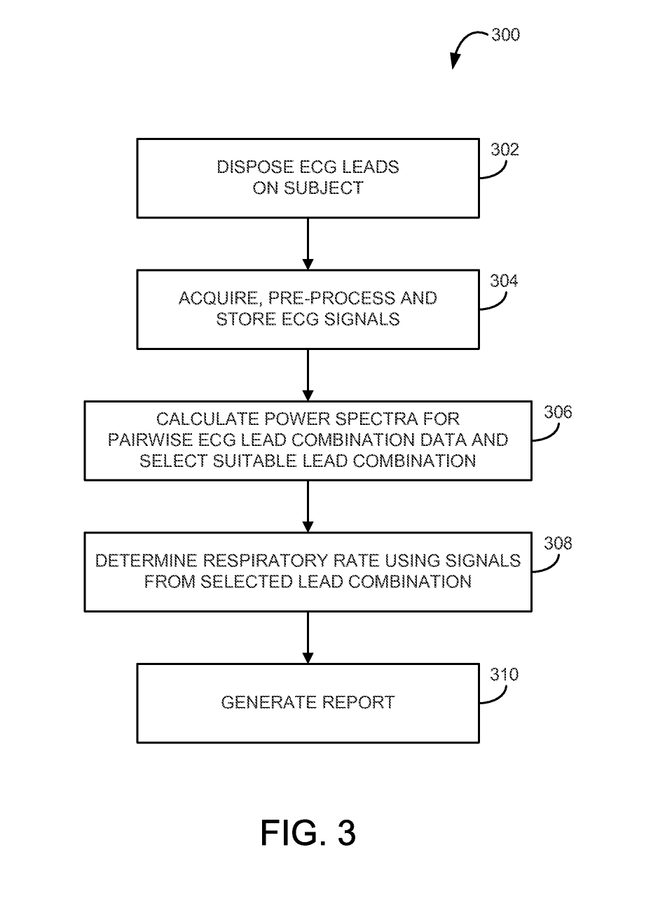

Turning to FIG. 3, a process 300 in accordance with the present disclosure begins at process block 302, whereby a series of ECG leads are disposed on a subject. The ECG lead configuration may include orthogonal and non-orthogonal lead combinations and, in some instances, may have no substantially orthogonal lead combinations. At process block 304, ECG signals may be acquired, pre-processed, and, if desired, stored into memory, or simply reported either continuously or intermittently. Pre-processing may involve any number of process steps, such as filtering and time-alignment. Then, at the next process block 306, power spectra are calculated based upon pair-wise ECG lead combinations and a desired combination is selected.

Once a suitable lead combination has been identified, the dominant power spectral peak determined, as will be described, that lead combination is then utilized to determine the RR at process block 308. And a report 310 is generated regarding the determined RR, which may take any desired shape, form or medium. For example, the report may include a displayed waveform, printed report, or other feedback.

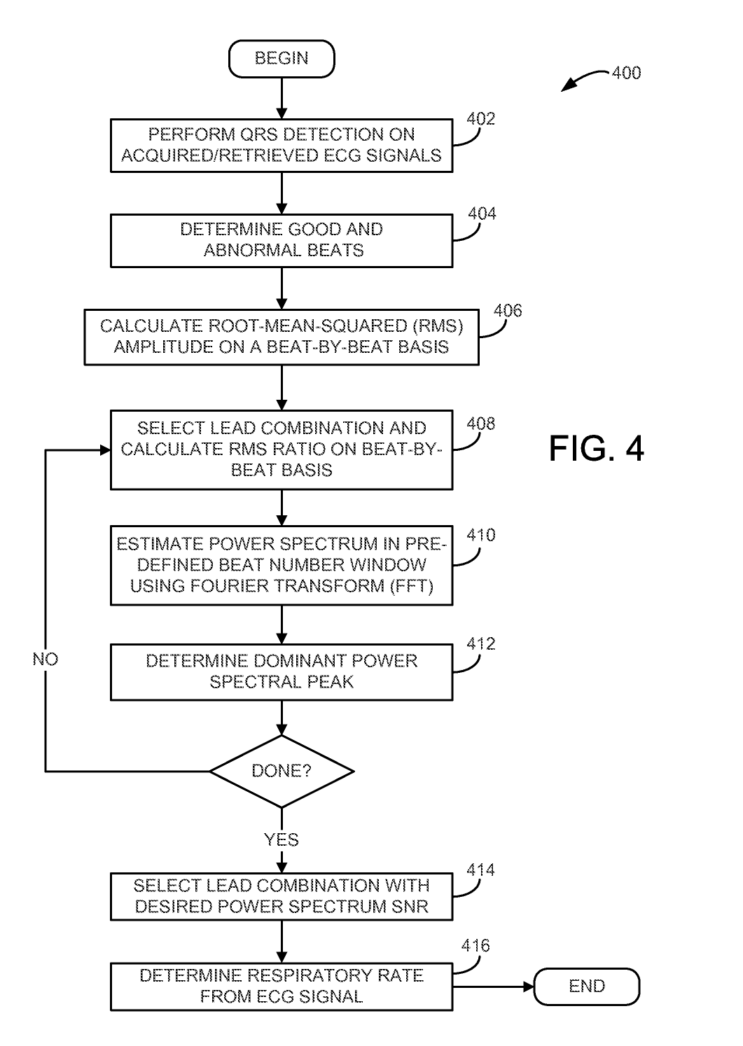

Turning to FIG. 4, the steps of a process 400 are provided for determining a desired or optimal ECG lead combination from a collection of ECG leads that include non-orthogonal lead combinations between leads that are generally required to be orthogonal. The process begins at process block 402, whereby preliminary R-wave annotations are obtained by applying a QRS detection algorithm to acquired or retrieved ECG signal data, for example, from surface electrogram lead V4. Then at process block 404, preliminary QRS detections are refined and abnormal beats, e.g. premature ventricular complexes (PVCs) and aberrantly conducted beats, are identified using a template-matching QRS alignment algorithm.

For each new beat, an exemplary 80 ms window centered at the peak of the QRS complex is formed from the preliminary R-wave detection. An isoelectric PR segment may be automatically subtracted as a zero amplitude reference point (by estimating the mean voltage in, for example, a 10 ms window preceding the start of each QRS complex). Then, a median QRS template is generated from all ‘normal’ QRS complexes in a sequence with predefined number of beats, and the current beat is aligned to the QRS template using cross-correlation. Cross-correlation may be repeated, for example, twice (or more), for each new QRS complex to ensure proper QRS alignment. A beat may be considered ‘abnormal’ if its correlation coefficient is less than, for a example a threshold value of 0.95 or if the preceding R-to-R interval is at least 10 percent shorter than the mean R-to-R interval of the previous, for example, 7 beats.

Next, at process block 406 the root-mean-squared (RMS) amplitude of each good beat may be calculated for all leads on a beat-by-beat basis using, for example, an 80 ms window centered at the QRS complex. The RMS amplitudes for all abnormal beats may be generated from neighboring RMS amplitudes using cubic-spline interpolation. By replacing aberrant beats with interpolated points, rather than the RMS values of the average good beats, discontinuities in the RMS ratio sequence prior to spectral analysis may be minimized. Next, at process block 408, a lead pair combination may be selected and an RMS amplitude ratio may be calculated on a beat-by-beat basis. Each ECG lead pair combination consists of a test ECG lead (the numerator), and a reference ECG lead (the denominator).

Thereafter, at process block 410 the power-spectrum in a predefined beat-number window of RMS ratio data is estimated using, for example a 512-length Fourier transform (FFT), to improve the frequency-domain resolution. The resulting frequency axis, in respiration cycles/beat (e.g. a range: 0-0.5 cycles/beat), may be converted to respirations per minute by scaling the axis, by for example, the average heart rate across the predefined beat-number window. At process block 412, the dominant power spectral peak is determined. If the dominant power spectral peak is found to be below, for example, 0.03 cycles/beat then the respiratory rate can be considered to be zero, corresponding to an apnic event. Alternatively, the dominant power spectral peak is found to be typically between 3 and 35 breaths/min, which corresponds to the detected RR for a selected ECG lead combination. The process is then repeated for the next selected lead combination, until all desired combinations have been evaluated.

When determining the desired or optimal lead combination at process block 414, the lead combination with the largest spectral signal-to-noise ratio (SNR) may be identified, whereby the SNR is defined as the spectral peak power divided by the median of the power spectrum from 0-0.5 cycles/beat, expressed in dB:

$$SNR = 10 {log}_{10} ( \frac{signal}{noise} ) \qquad ( 1 )$$

This method provides one sample of the ECG-derived respiration per cardiac cycle. Given that the heart rate is almost always greater than twice the RR, the RR can be measured well from this limited set of samples.

Thereafter, at process block 416, respiratory rate can be estimated from any two electrocardiographic leads, for example, by finding the power spectral peak of the derived ratio of the estimated root-mean-squared amplitude of the QRS complexes on a beat by beat basis across a 32-beat window, and automatically selecting the lead combination with the highest power spectral signal-to-noise ratio.

Alternatively, to overcome the occurrence of frequent PVCs, for a beat sequence of, for example, 32 beats, that includes, for example, less than 90% good beats, the respiratory rate may be estimated through interpolation of the respiratory rate values of neighboring beat sequences that include more than 90% good beats.

### EXAMPLE

In ten mechanically ventilated swine we collected intracardiac electrograms from catheters in the right ventricle, coronary sinus, left ventricle, and epicardial surface, as well as body surface electrograms, while the ventilation rate was varied between 7 and 13 breaths/min at tidal volumes of 500 and 750 mL. We found excellent agreement between the estimated and true respiratory rate for right ventricular (R²=0.97), coronary sinus (R²=0.96), left ventricular (R²=0.96), and epicardial (R²=0.97) intracardiac leads referenced to surface lead ECGII. When applied to intracardiac RV-CS bipolar leads, the algorithm exhibited an accuracy of 99.1 percent (R²=0.97). When applied to 12-lead body surface ECGs collected in four swine, the algorithm exhibited an accuracy of 100 percent (R²=0.93). In conclusion, the present algorithm provided an accurate estimation of the respiratory rate using either intracardiac or body surface signals, without the need for additional hardware.

### Animal Preparation

Ten male Yorkshire swine (40-45 kg) were anesthetized and acutely instrumented in the Animal Electrophysiology Laboratory of the Massachusetts General Hospital. Anesthesia was induced with Telazol (4.4 mg/kg) im and Xylazine (2.2 mg/kg) im. Each animal was intubated and placed on a mechanical ventilator, and anesthesia was maintained with Isoflurane (1.5-5 percent).

Percutaneous access was achieved by inserting standard angiographic sheaths into the femoral arteries and veins using Seldinger technique (28, 33). Decapolar catheters were placed under fluoroscopic guidance in the right ventricle (RV, the distal lead being in the RV apex), coronary sinus (CS, the distal lead being in the distal CS), left ventricle (LV, the proximal lead being in the LV apex), and the ventricular epicardial space (EPI). Epicardial access was achieved utilizing a standard sub-xyphoid percutaneous approach (as it is typically clinically performed in humans) (7, 8). Briefly, a sheath was placed into the pericardial space using a Tuohy needle. Then the catheter was maneuvered into the space through this sheath. Finally, an inferior vena cava catheter was inserted as a reference electrode for unipolar signals and the actual locations of the catheters were verified by 2D x-ray views of the heart. Traditional electrocardiographic (ECG) electrodes were placed on the animal's limbs and chest.

### Data Recording Equipment

Body surface ECG and intracardiac EGM signals were recorded through a Prucka Cardiolab (Generic Electric) electrophysiology system that provided 16 high fidelity analog output signals and front-end signal conditioning. Body surface signals were band-pass filtered 0.05-100 Hz, with 60 Hz notch filter and gain 2500 V/V, and intracardiac signals were band-pass filtered 0.05-500 Hz, with 60 Hz notch filter and gain 250 V/V.

We have recently developed a state-of-the-art signal acquisition, display and processing system, which supports the acquisition, display, and real-time analysis of all 16 Prucka output signals, sampled at 1000 Hz by a multi-channel 16-bit data acquisition card (National Instruments M-Series PCI6255). This system includes custom software written in LabView 8.5 (National Instruments, Austin, Tex.) and MATLAB 7.6 (Mathworks, Natick, Mass.). This system was modified to estimate and display the RR in real-time using either body surface ECG and/or intracardiac EGM signals.

A respiratory monitor (Surgivet V9004) was used as the gold standard to measure the RR throughout each respiratory intervention. This monitor has an accuracy of ±1 breath/min, and functions as follows: each respiration event is detected at the leading edge (upswing) of the CO₂ waveform; next, each set of 4 consecutive breaths is averaged using box-car averaging; finally, the RR is rounded down and displayed by the unit.

### Data Collection

For each mechanically ventilated animal, body surface and intracardiac EGMs were recorded while the ventilation rate was stepped from 13 to 7 breaths/min, at tidal volumes of 500 mL and 750 mL. Each ventilation rate was maintained for a minimum of 90 seconds.

In the intracardiac recording configuration, electrogram signals were recorded from two body surface leads (lead II and V4) and 12 intracardiac unipolar leads, including three leads from the RV catheter (RV1, RV2, and RV7, where “1” is the most distal electrode), three leads from the CS catheter (CS1, CS2, and CS7), three leads from the LV catheter (LV1, LV2, and LV9), and three leads from the EPI catheter (EPI1, EPI2, and EPI9). All unipolar leads were referenced to the same lead in the inferior vena cava catheter. Bipolar intracardiac leads were reconstructed by subtracting pairs of unipolar leads, including four far-field bipolar leads (RV71, CS71, LV91, and EPI91), four near-field bipolar leads (RV21, CS21, LV21, and EPI21) and two inter-catheter bipolar leads (RV1CS1 and RV1CS7). A set of intracardiac recordings was collected in 8 animals, and a set of 12-lead body surface ECG recordings was collected in 4 animals.

### Results

### Determination of the Optimal Beat Window Length

We first sought to determine the optimal number of beats on which to perform FFT analysis (the beat window) that would maximize the accuracy and minimize the latency required to estimate the RR.

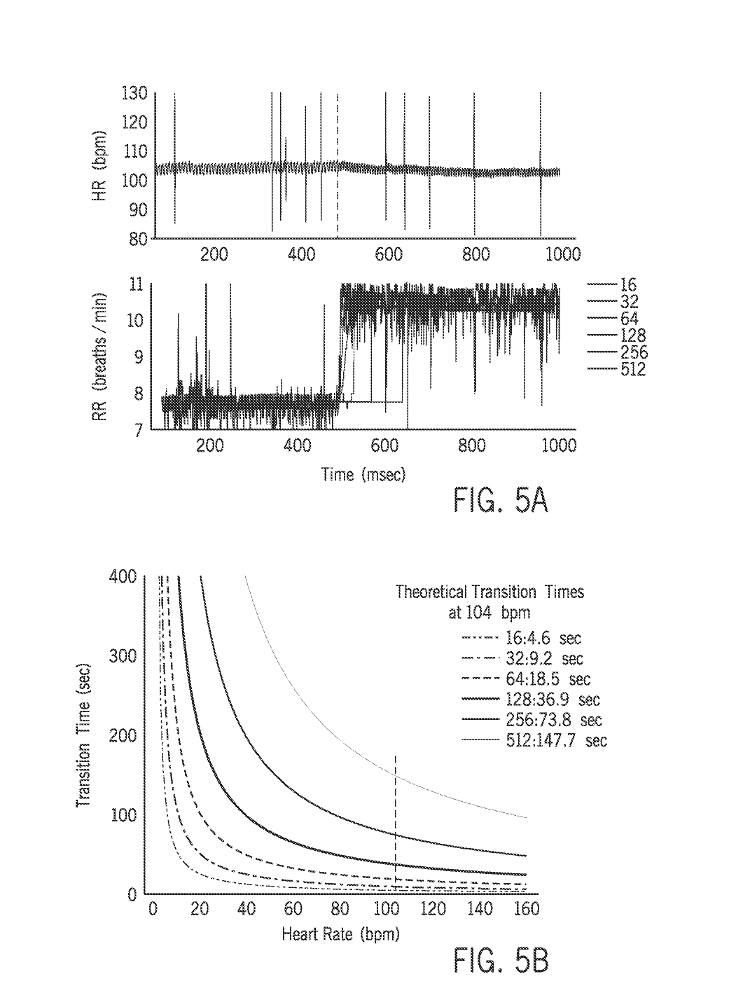

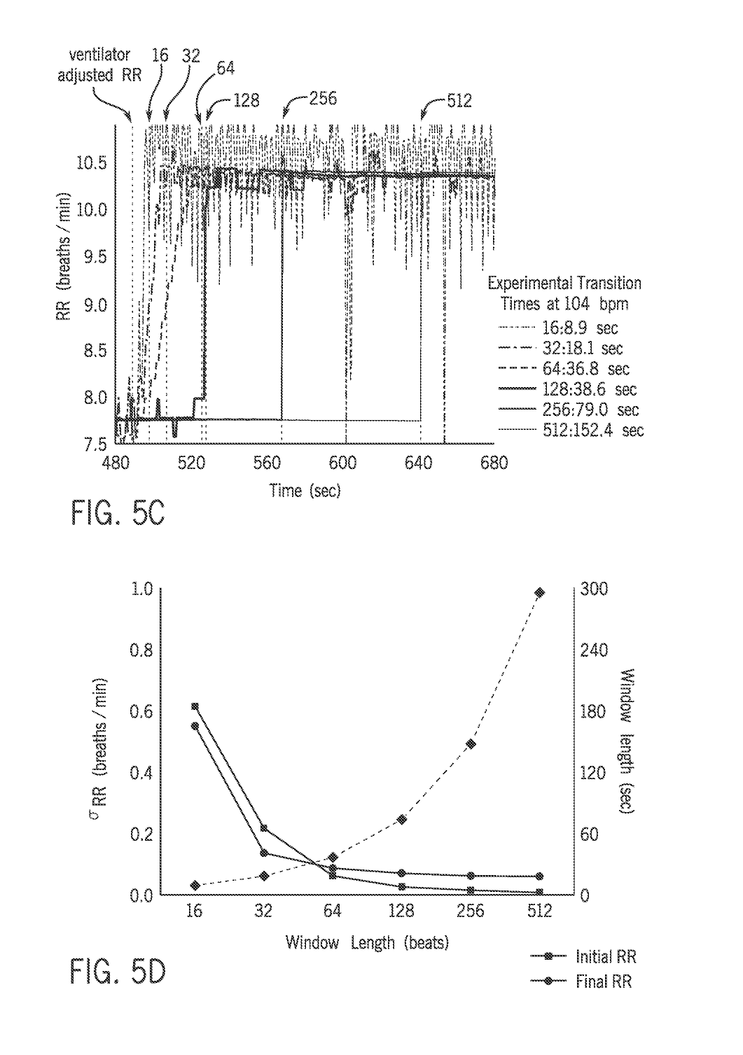

In FIG. 5A we show the heart rate and respiration rate of a recording in which the ventilator's rate was changed from 7 breaths/min to 10 breaths/min, 489 sec after the beginning of the recording (the dotted line indicates the timing of the change in the ventilator frequency). We estimated the respiratory rate using a 16-, 32-, 64-, 128-, 256-and 512-beat window. In FIG. 5B we present the theoretical estimation of the transition time required for each of the 16-, 32-, 64-, 128-, 256-and 512-beat windows to reach a new rate (window length in beats * 60/Heart Rate in bpm/2). Given that at 489 sec the instantaneous heart rate was 104 bpm, the theoretical transition times were 4.6, 9.2, 18.5, 36.9, 73.8 and 147.7 sec, respectively (the dotted vertical line indicates 104 bpm). In FIG. 5C we present the estimated respiratory rate plotted as a function of time; the data are fitted with the Boltzmann equation (y =A2 +(A1⁻A2)/(1 +exp((x-x₀)/dx))) to obtain the experimental transition times of 8.9, 18.1, 36.6, 38.8, 79.0 and 152.4 sec, respectively. We observe that the theoretical transition times predicted are in excellent agreement with the estimated values of FIG. 5C. In FIG. 5D we show the standard deviation of the respiratory rate estimates using a 16-, 32-, 64-, 128-, 256-and 512-beat window (left axis); we also show the window length (in time). We observe that for RR estimation error of less than one breath per minute, the 32-beat window provides an uncertainty that is less than 0.5 beats per minute. Thus, although the RR estimation error is smaller with a larger size window, the benefit of the increased accuracy is not substantial to justify the more than doubling of the number of beats required to correctly estimate the RR. Therefore, in the remainder of this study we used a 32-beat window.

### Algorithm Demonstration

To examine the ability of our algorithm (without the optimization step) to accurately estimate the RR we performed a series of experiments in which the ventilator rate was adjusted in either a step-down or step-up fashion, from 7 to 13 breaths/min. The respiratory monitor output (as displayed on the monitor screen) was recorded throughout each experiment to serve as the gold standard to evaluate our algorithm's accuracy during time intervals at which the RR was held constant.

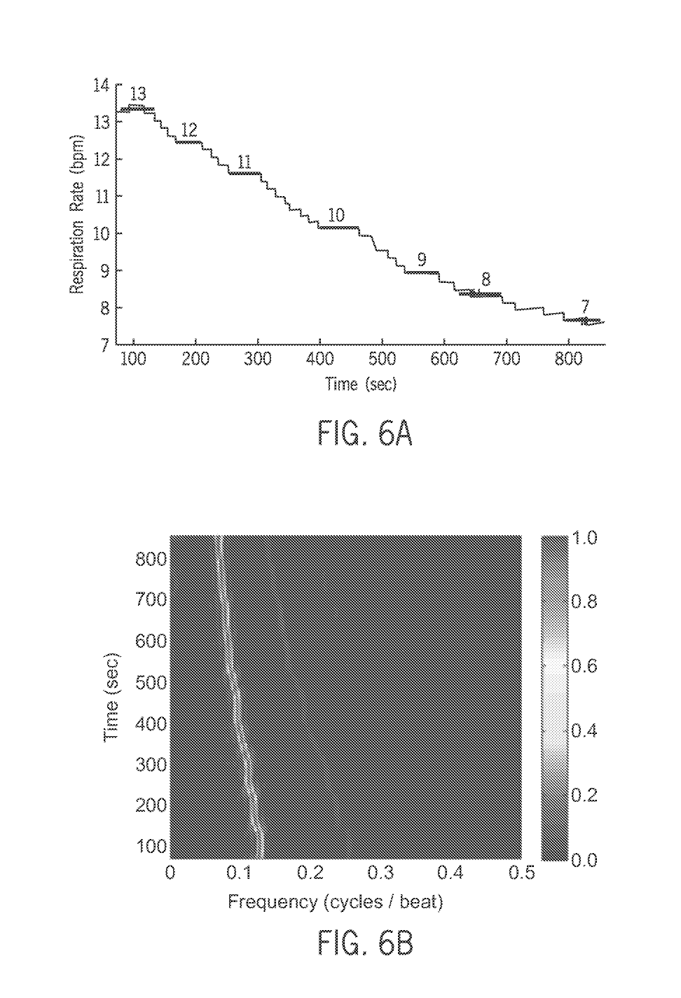

In FIG. 6A, we show a representative example of this process, in which the ventilator was stepped down from 13 to 7 breaths/min. The blue line indicates the estimated RR (here using the most distal CS lead, CS1, referenced to ECG lead II) throughout the time-course of the recording, while the red lines show the RR reported by the respiratory monitor during the time intervals at which the RR is held steady and the algorithm reports a constant RR. This process was repeated for all leads in each study.

In FIG. 6B, we show the normalized power spectrum (in cycles/beat) as a function of time during the step down transition of the ventilator's rate, from 13 to 7 breaths/min. We see that there is a clear peak at 0.128 cycles/beat (at a heart rate of 104 bpm) in the spectrum corresponding to 13 breaths/min which progressively moves with every new ventilator RR setting to a final peak at 7 breaths/min.

### Estimation of Respiratory Rate Using Intracardiac Leads

We next examined the ability of the algorithm (without the optimization step) to estimate the RR using unipolar, far-field bipolar, near-field bipolar and RV-CS intracardiac leads. For this analysis, each intracardiac lead (numerator) was referenced to body surface ECG lead II (denominator) to maximize the potential for ratiometric lead orthogonality. The absolute error and percent of missed detections using each intracardiac lead configuration were calculated for each animal across all ventilation rates, from 13 to 7 breaths/min, at tidal volumes of 500 mL and 750 mL.

The absolute error was calculated as the average difference between the estimated and true RR. A missed detection was defined as an RR detection in which the estimated RR differed from the true RR by more than one breath/min (the accuracy of the respiration monitor), that is, |Estimated RR-True RRA|>1. Because the respiratory monitor rounds each RR down to the nearest integer, each estimated RR was also rounded down to the nearest integer prior to absolute error and missed detection calculation. For each lead, differences between tidal volume 500 mL and 750 mL were quantified using a non-parametric paired Wilcoxon signed rank test, and differences across leads were quantified using a multiple comparison test from a one-way ANOVA test, with both tests rejecting the null hypothesis at p <0.05.

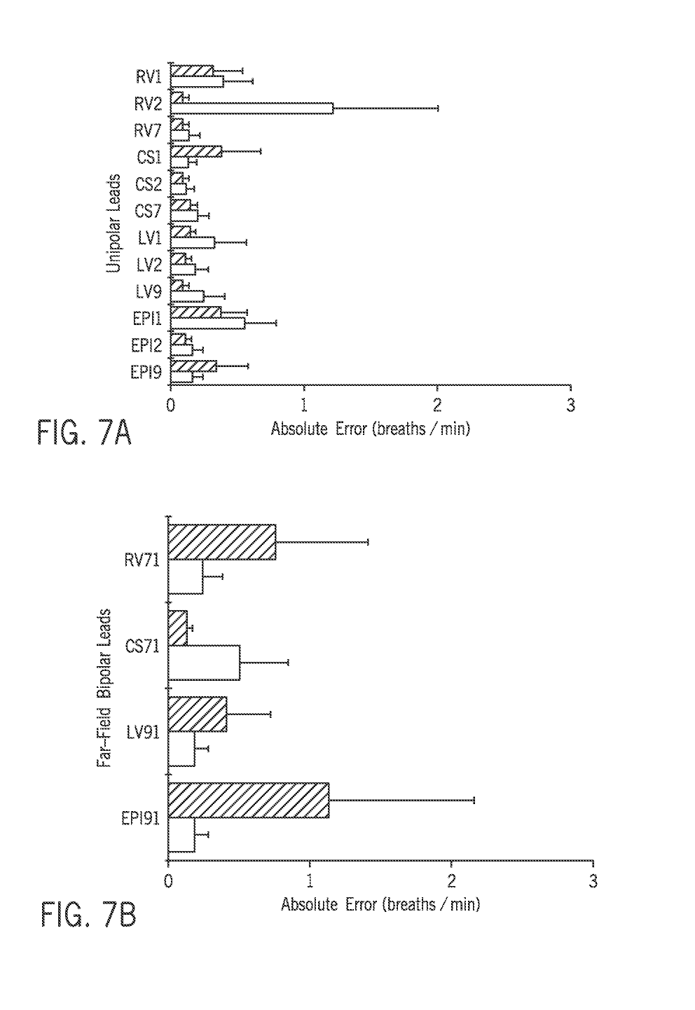

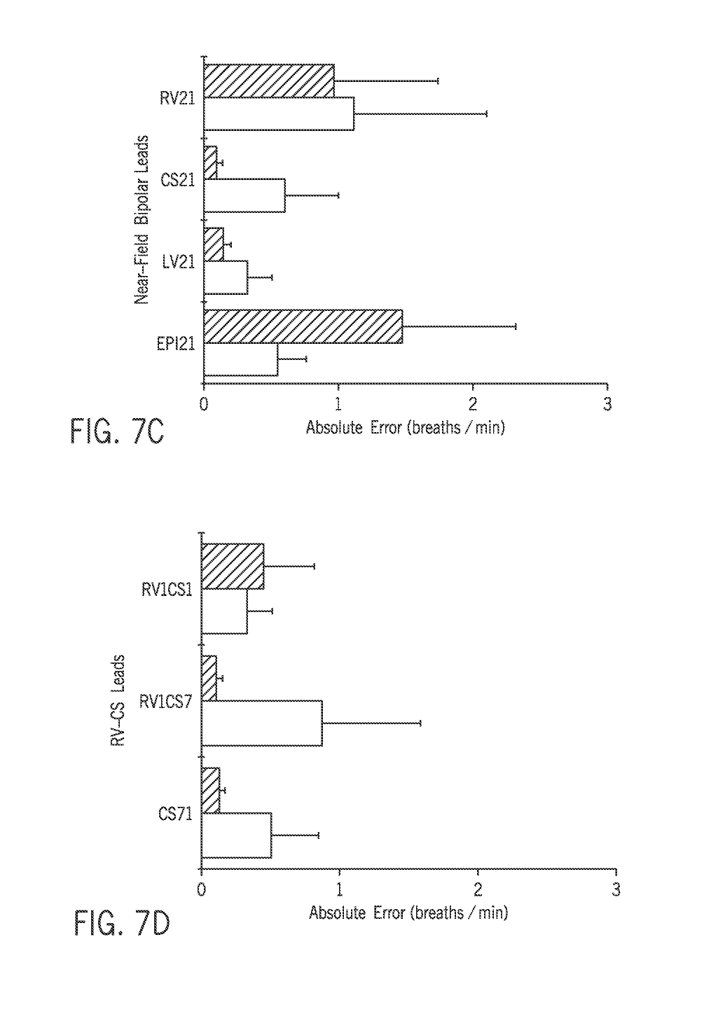

In FIG. 7, we show the absolute error for each lead at tidal volumes of 500 and 750 mL, averaged across all animals. FIG. 7A shows results from unipolar leads, FIG. 7B shows results from far-field bipolar leads, FIG. 7C shows results from near-field bipolar leads, and FIG. 7D shows results from RV-CS intercatheter leads. No statistical difference of the error was found between any paired tidal volume comparison for any intracardiac lead, and no statistical difference was found between any two far-field bipolar, any two near-field bipolar, or any two RV-CS leads, respectively.

In FIG. 7A, we observe that the absolute error for unipolar leads has a range of 0.09-1.22 breaths/min, with a mean of 0.26 breaths/min. No statistical difference was found between any unipolar leads except lead RV2 at tidal volume 750 mL, which was greater than 13 other intracardiac lead tests. In FIG. 7B, we observe that the absolute error for far-field bipolar leads has a range of 0.13-1.13 breaths/min, with a mean of 0.44 breaths/min. FIG. 7C demonstrates that the absolute error for near-field bipolar leads has a range of 0.09-1.47 breaths/min, with a mean of 0.66 breaths/min and FIG. 7D demonstrates an absolute error of 0.11-0.87 breaths/min with a mean of 0.40 breaths/min for RV-CS bipolar leads. As shown by the small absolute errors in all intracardiac lead configurations, the algorithm closely tracks the true RR across a wide range of intracardiac leads.

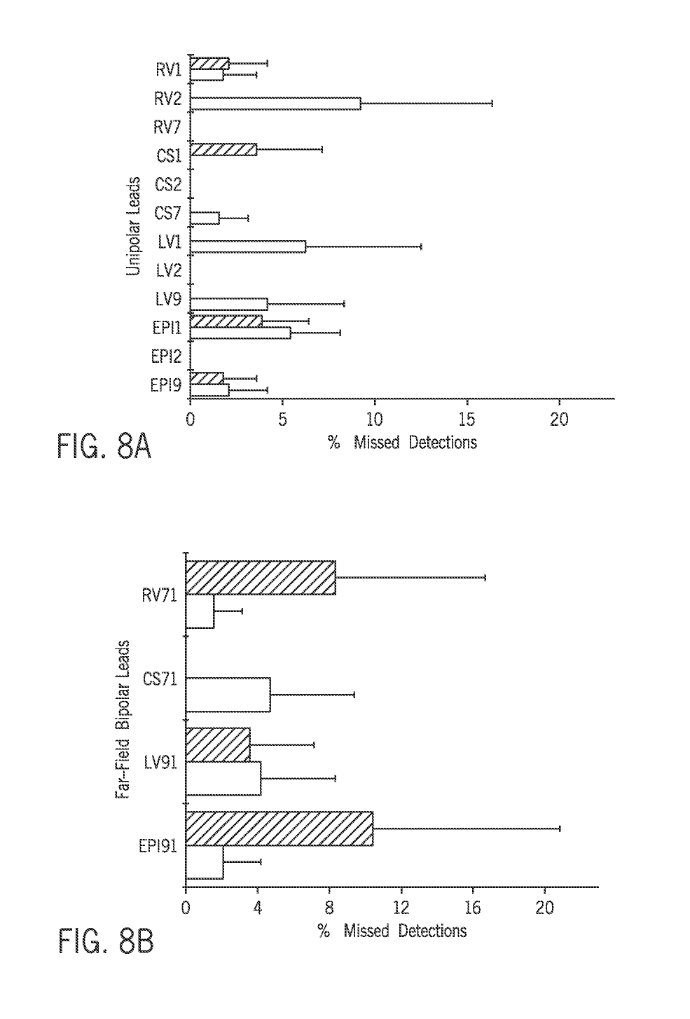

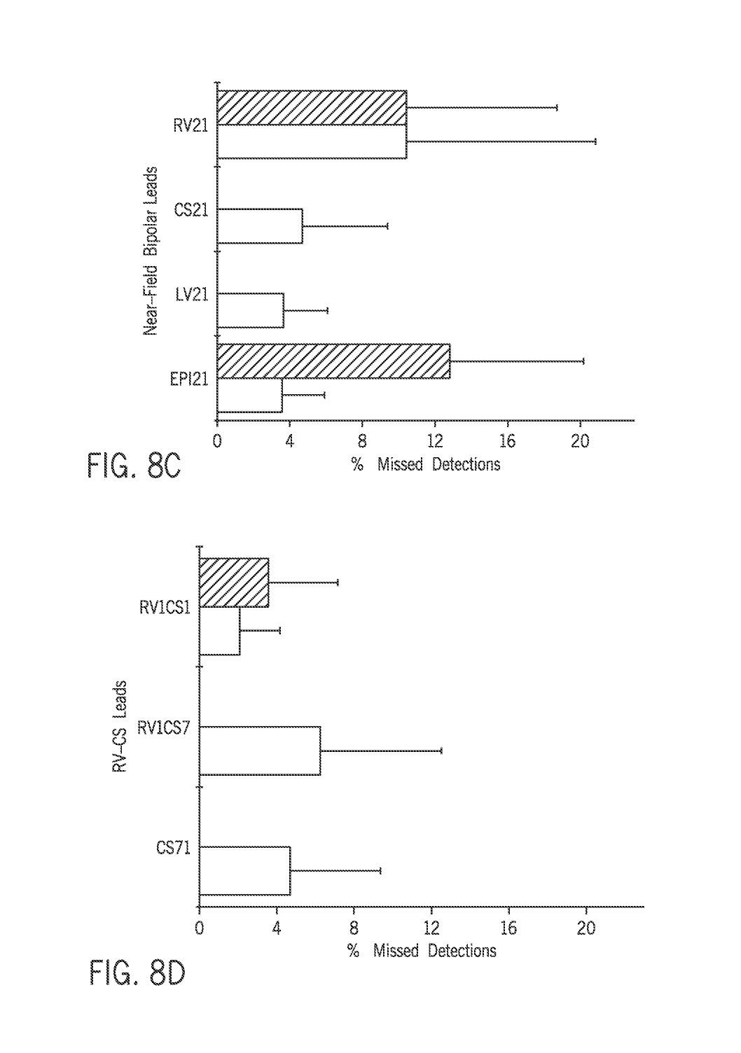

In FIG. 8, we show the percent of missed detections for each lead at tidal volumes of 500 and 750 mL, averaged across all animals. FIG. 8A shows results from unipolar leads, FIG. 8B shows results from far-field bipolar leads, FIG. 8C shows results from near-field bipolar leads, and FIG. 8D shows results from RV-CS leads. No statistical difference was found for the percent of missed detections between any paired tidal volume comparison for any intracardiac lead, and no statistical difference was found between any two unipolar, any two far-field bipolar, any two near-field bipolar, or any two RV-CS leads, respectively.

In FIG. 8A, we observe that the percent of missed detections for unipolar leads has a range of 0.0-9.2 percent, with a mean of 1.7 percent. In FIG. 8B, the percent of missed detections for far-field bipolar leads also has a range of 0.0-9.2 percent, with a mean of 1.7 percent. FIG. 8C demonstrates that the percent of missed detections for near-field bipolar leads has a range of 0.0-12.9 percent, with a mean of 5.7 percent and finally, FIG. 8D demonstrates that the percent of missed detections for RV-CS bipolar leads has a range of 0.0-6.3 percent, with a mean of 2.8 percent. While the average percent of missed detections in all intracardiac lead configurations is low, the maximum percent of missed detections on select leads is higher than desired for a robust RR detection algorithm.

### Lead Optimization

To examine the conditions leading to the failure of the proposed algorithm to accurately estimate RR, we compared the spectral SNR of all accurate versus missed RR detections for all unipolar, far-field bipolar, near-field bipolar and RV-CS intracardiac leads, referenced to surface ECG lead II.

The spectral SNR using each intracardiac lead configuration was compiled across all animals, tidal volumes and ventilation rates. The SNR of all accurate versus missed RR detections were then compared for every intracardiac lead type using a non-parametric Mann-Whitney U-test

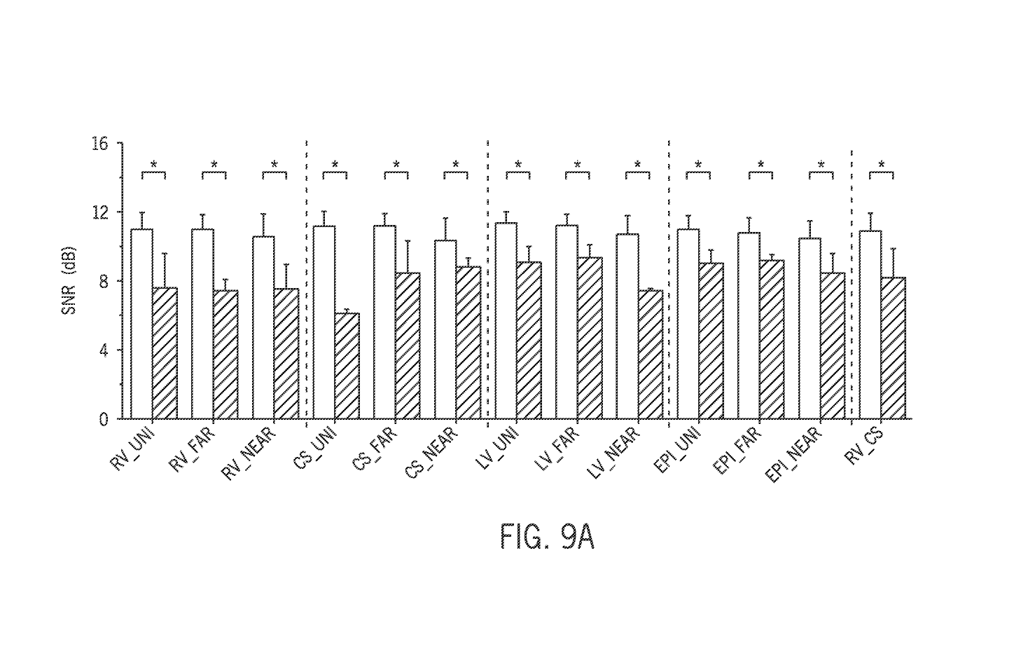

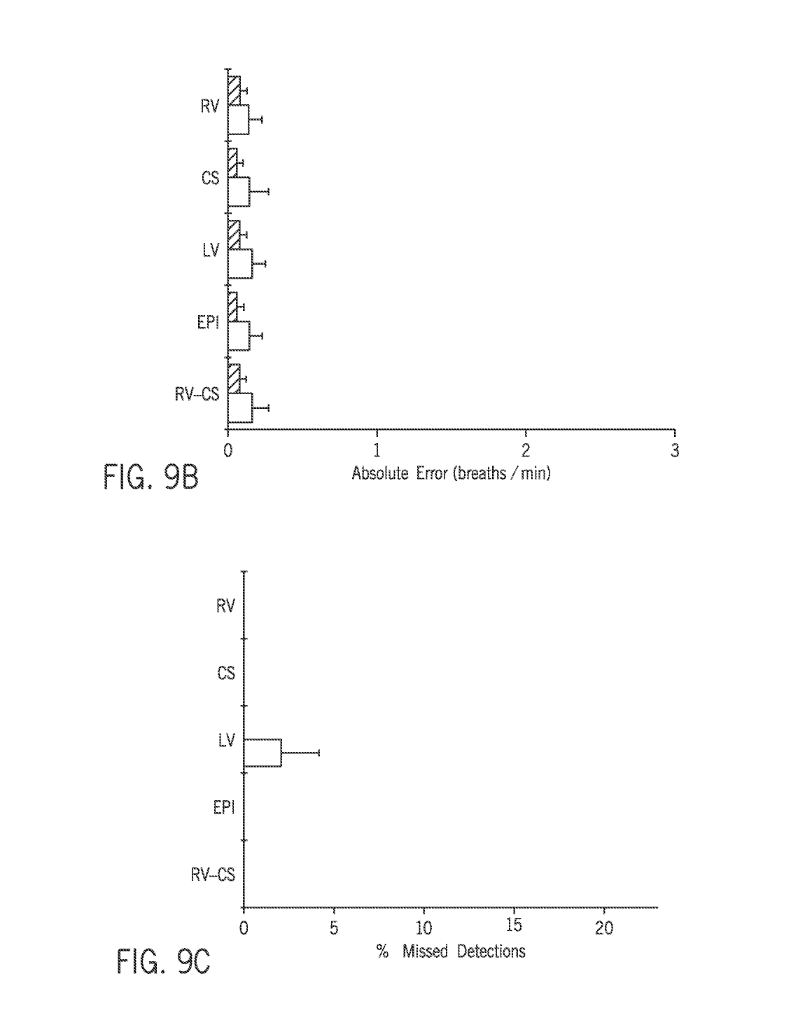

In FIG. 9A we plot the mean and standard deviation of the spectral SNR for every intracardiac lead combination for all accurate and missed detections. For each lead type, the accurate detection SNR is significantly larger than the missed detection SNR (all p<0.034). Across all lead types, the average accurate detection SNR is 11.0 dB, and the average missed detection SNR is 8.2 dB.

We next grouped all intracardiac leads by catheter type (RV, CS, LV, EPI, or RV-CS, grouping all unipolar, far-field bipolar, and near-field bipolar leads from the same catheter), and re-analyzed the data using our optimized algorithm. For each catheter, the lead combination with the highest SNR at each ventilation rate was used to estimate the RR. In FIG. 9B we plot the absolute error for each catheter at tidal volumes of 500 mL and 750 mL, averaged across all animals. The absolute error using our optimized algorithm has a range of 0.09-0.16 breaths/min, with a mean of 0.13 breaths/min. In FIG. 9C we plot the percent of missed detections for each catheter at tidal volumes of 500 mL and 750 mL, averaged across all animals. The percent of missed detections using our optimized algorithm has a range of 0.0-2.1 percent, with a mean of 0.2 percent. There were no missed detections using the RV, CS, EPI and RV-CS leads. Notably, the only missed detection came from a single RR measurement in a single animal at tidal volume 750 ml in which the maximum SNR was less than 7 dB for the LV lead grouping.

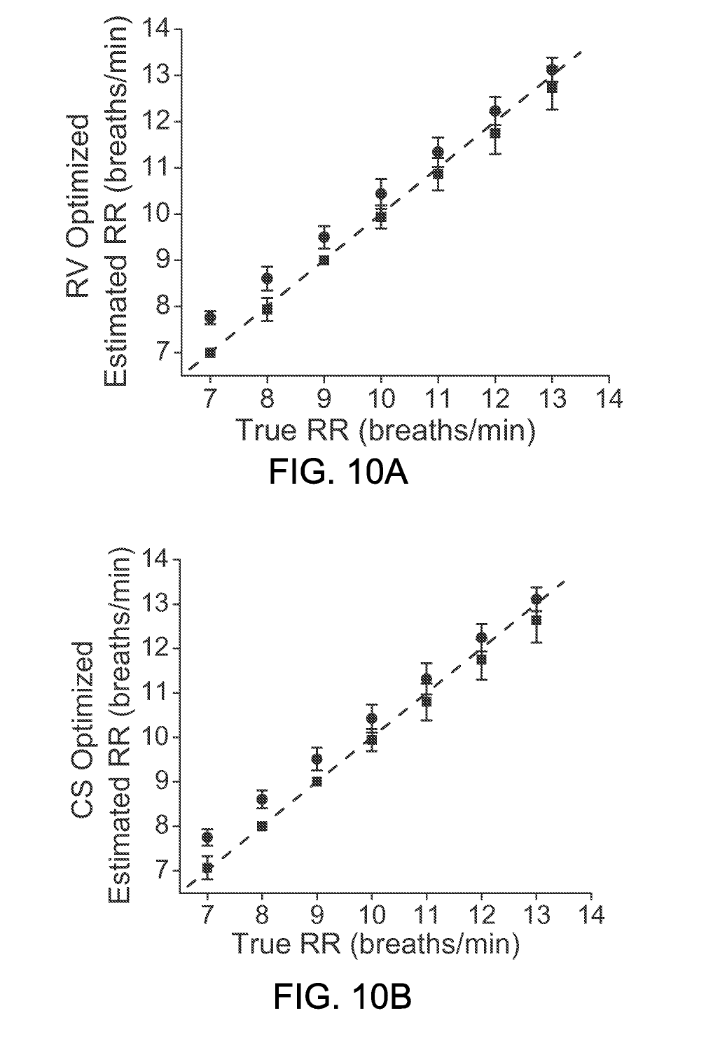

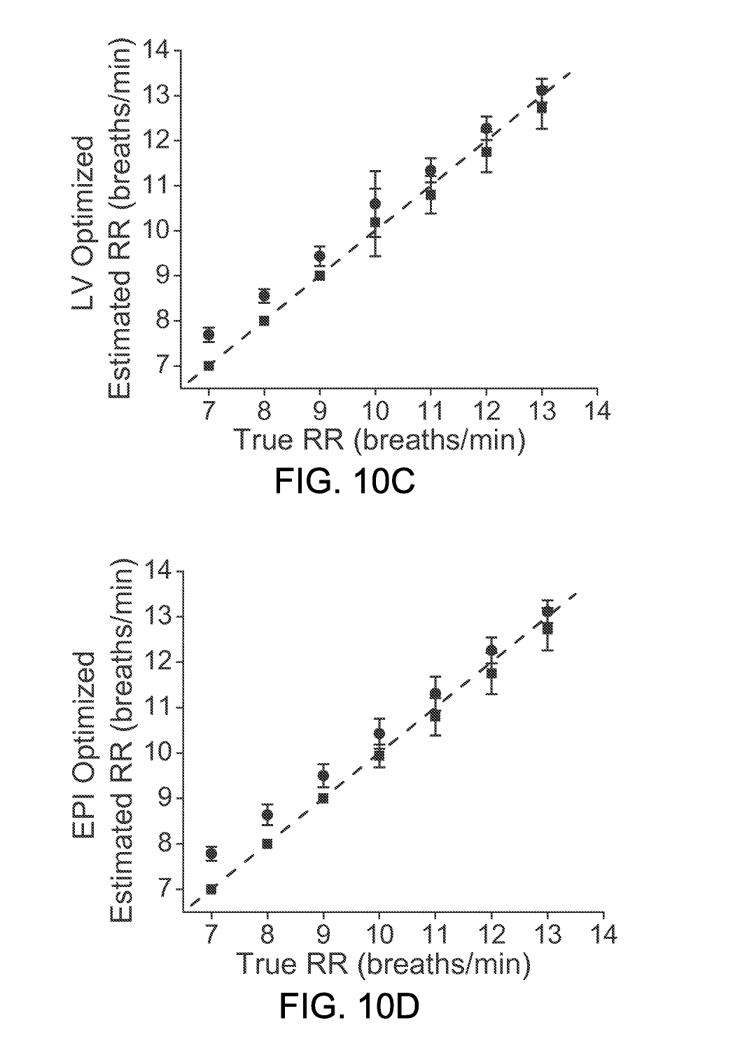

We further characterized the performance of our optimized algorithm by calculating the goodness of fit between the estimated and true RR across all ventilation rates. In FIG. 10 we plot the estimated versus the true RR in FIG. 10A RV, in FIG. 10B CS, in FIG. 10C LV, and in FIG. 10D EPI lead groupings for both non-rounded and down-rounded RR estimates, compiled across all animals and tidal volumes. The rounded RR estimates closely track the true RR, with goodness-of-fit R² statistic of 0.97, 0.96, 0.96, 0.97, and 0.96 for RV, CS, LV, EPI, and RV-CS estimates, respectively (RV-CS data not shown).

### Optimized Intracardiac Respiratory Rate Estimation Using RV-CS Bipolar Leads

To develop an RR estimation method that could be deployed in an intracardiac device and which does not rely on the use of any surface ECG lead, we evaluated the performance of our optimized algorithm using ratiometric combinations of bipolar leads RV1CS1, RV1CS7, and CS1CS7.

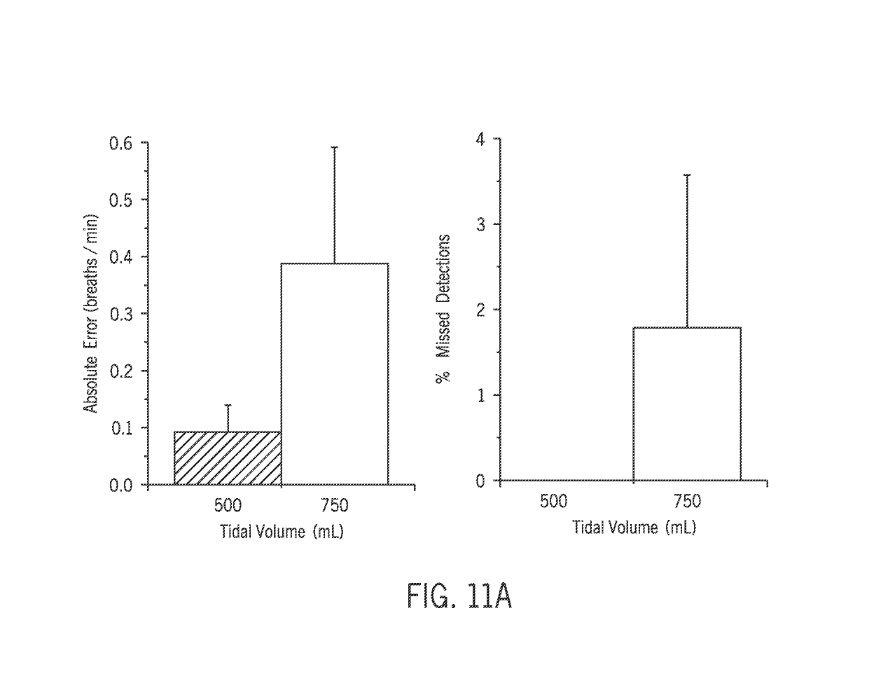

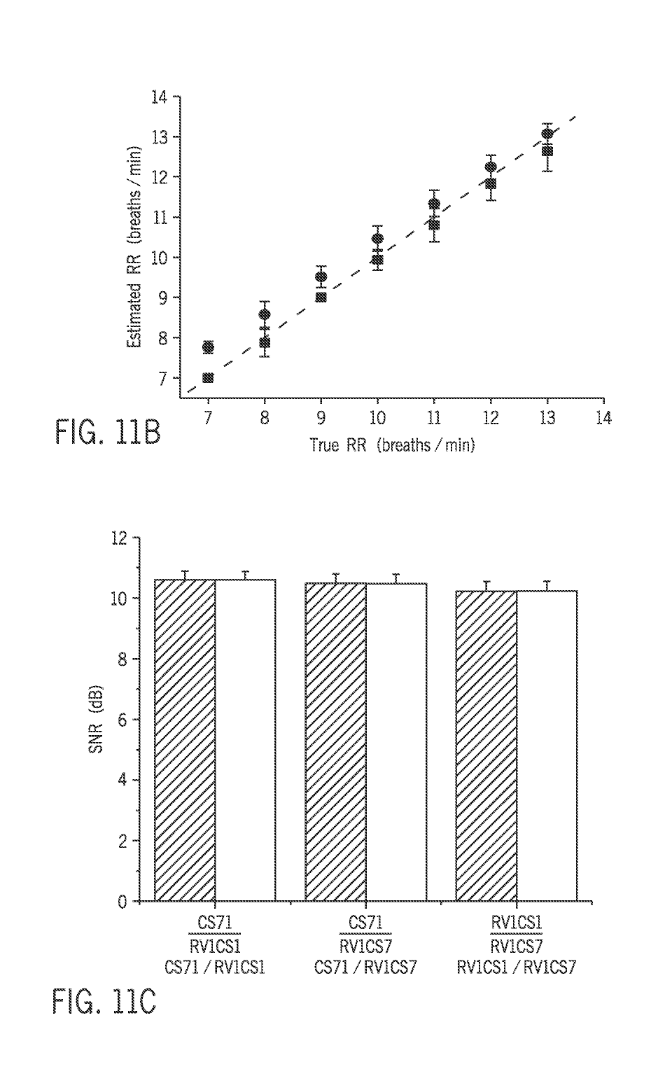

In FIG. 11A we show the absolute error and percent of missed detections at tidal volumes of 500 and 750 mL, averaged across all animals. This method is highly accurate when applied to intracardiac-only RV-CS bipolar leads, with an average absolute error of 0.09 and 0.39 breaths/min, respectively, at tidal volume 500 mL and 750 mL, and a missed detection percentage of 0 percent and 1.78 percent, respectively, at tidal volume 500 mL and 750 mL. Only one RR intervention was improperly detected. In FIG. 11B we plot the estimated versus true RR across all ventilation rates for both non-rounded and down-rounded RR estimates, compiled across all animals and tidal volumes. The rounded RR estimates closely track the true RR, with goodness-of-fit R² statistic of 0.97. In FIG. 11C we plot the average SNR of the six RV-CS lead combinations. While no statistical difference was found between any pair of lead combinations (using a paired Wilcoxon signed rank test), the CS71/RV1CS1 lead combinations trended higher than the RV1CS1/CS71 lead combinations, which trended higher than the CS71/RV1CS7 lead combinations.

Indeed, 42.86 percent of the optimized lead configurations use a combination of the CS71 and RV1CS1 leads, 32.38 percent of the optimized lead configurations use a combination of the RV1CS1 and CS71 leads, and 24.76 percent of the optimized lead configurations use a combination of the CS71 and RV1CS7 leads. With only one missed detection, the overall accuracy of this intracardiac algorithm is 99.1 percent.

Estimation of the Respiratory Rate from Body Surface Signals

To further evaluate this method in estimating the RR, we applied this method on 12-lead ECG recordings in 4 animals. We estimated the RR by obtaining for each 32 beat sequence the ratio of any two body surface leads that provided the highest SNR. We found that this method provided 100 percent accurate estimation of the RR, with no missed detections.

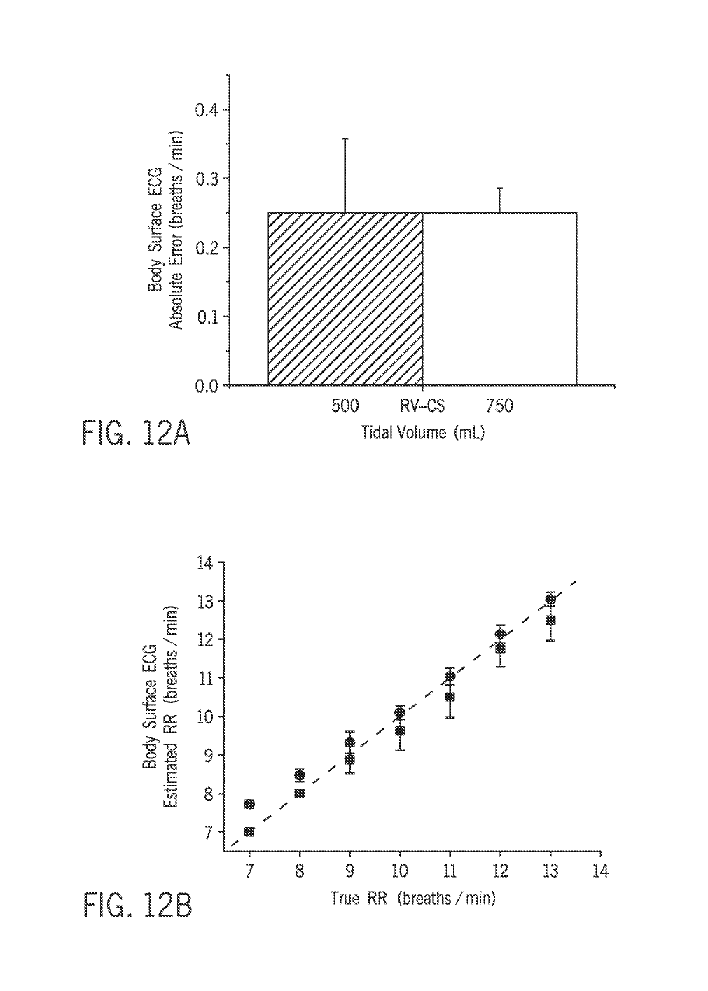

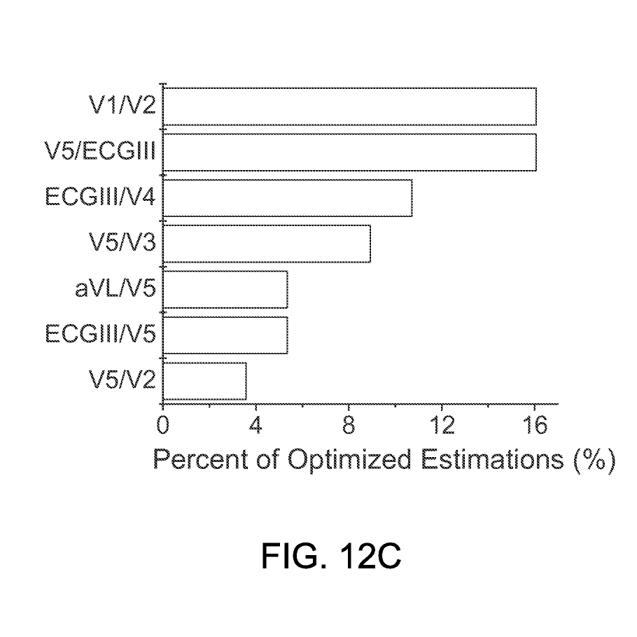

In FIG. 12A we show the absolute error at tidal volumes of 500 and 750 mL, averaged across all animals. This method exhibits an average absolute error of 0.25 and 0.25 breaths/min, respectively, at tidal volume 500 mL and 750 mL. In FIG. 12B we plot the estimated versus true RR across all ventilation rates for both non-rounded and down-rounded RR estimates, compiled across all animals and tidal volumes. The rounded RR estimates closely track the true RR, with goodness-of-fit R² statistic of 0.93. In FIG. 12C we identify the seven most-used lead configurations by the optimized algorithm. Ratiometric configurations V1/V2 and V5/ECGIII were each used 16.1 percent of the time, followed by ECGIII/V4 (10.7 percent), V5/V3 (8.9 percent), aVL/V5 (5.4 percent), ECGIII/V5 (5.4 percent), and V5/V2 (3.6 percent). Of note, the pairing of ECGIII and V5 was the most commonly selected pairing, accounting for 19.6 percent of all optimized pairings.

In this study we implemented a novel algorithm to accurately and efficiently estimate the respiratory rate from either intracardiac or body surface leads. Overall, we have shown that the presented method first, does not require specialized hardware to measure the RR but rather uses only standard electrocardiographic signals; second, estimates RR with high accuracy utilizing either intracardiac or body surface electrocardiographic signals; and third, automatically optimizes the lead choice for real-time RR estimation without requiring any a priori knowledge of lead orthogonality.

For intracardiac RR estimation we presented a method that uses ratiometric combinations of bipolar ECG leads RV1CS1, RV1CS7, and CS1CS7. RV and CS catheters are commonly implanted with cardiac-resynchronization therapy (CRT) devices in heart failure patients, and these bipolar ECG lead configurations form a triangle, ensuring a range of angles between ratiometric lead pairs to optimize RR estimation. We found the overall accuracy of this intracardiac method to be 99.1 percent.

For RR estimation using 12-lead ECGs our algorithm was 100 percent accurate. The most commonly selected ECG leads for ratiometric RR estimation include frontal ECG lead III and precordial lead V5, at least one of which was automatically selected by our algorithm in six of the seven most-used ratiometric lead combinations, and together were used 19.6 percent of the time. This finding supports the possibility that only a subset of ECG leads is required for high-fidelity ambulatory ECG-based RR estimation, including only leads III and V5. Finally, the use of 32-beat window makes this algorithm suitable to respond to faster RR changes, as may be found with Cheyne-Stokes respiration. The trade-off for reducing the beat window length for insignificantly reduced accuracy, is not expected to affect the performance of this method (FIG. 5A-5C).

Thus, the proposed highly accurate and efficient algorithm takes advantage of simple hardware that is readily available as part of electrocardiographic patient monitoring to provide the RR as an additional physiological parameter that may help improve diagnosis, treatment and outcomes across a variety of clinical settings.

The present invention has been described in terms of one or more preferred embodiments, and it should be appreciated that many equivalents, alternatives, variations, and modifications, aside from those expressly stated, are possible and within the scope of the invention.

What is claimed is:

1. A method for determining respiratory rate of a subject from elctrocardiographic (ECG) signals acquired from the subject, the method comprising the steps of:
(a) acquiring ECG signals from an ECG monitor coupled to the subject through a plurality of ECG leads, wherein the plurality of ECG leads includes traditionally-orthogonal lead groups that are non-orthogonal;
(b) determining a combination of ECG leads from the plurality of ECG leads that provides a spectral signal-to-noise ratio (SNR) above a threshold value;
(c) processing the ECG signals from the combination of ECG leads determined in step (b) to extract a respiratory rate of the subject from the ECG signals; and
(d) generating a report indicating a respiratory rate of the subject determined based on step (c).

2. The method of claim 1, wherein at least one of step (b) and step (c) includes determining at least one of a series of root-mean-squared (RMS) amplitudes, consistent with R-wave occurrences in QRS complexes, from the ECG signals acquired for each of the plurality of ECG leads, on a beat-by-beat basis.

3. The method of claim 2, further comprising determining a series of RMS amplitude ratios using a first and a second series of RMS amplitudes for the plurality of ECG lead combinations, on a beat-by-beat basis.

4. The method of claim 3, further comprising determining a set of power spectra using a fast Fourier transform (FFT) of the series of RMS amplitude ratios in a pre-defined beat number window, for the plurality of ECG lead combinations.

5. The method of claim 4, wherein each of the set of power spectra is characterized by a SNR defined by:
$$SNR = 10 {log}_{10} ( \frac{signal}{noise} )$$
whereby the “signal” represents the spectral peak power and the “noise” is given by a median of the power spectrum, for a frequency ranging from 0 to 0.5 cycles/beat.

6. The method of claim 5, wherein a frequency axis for each of the set of power spectra is converted from a number of cycles per beat to a number of respirations per minute using a scale obtained from a heart rate estimate across a predefined beat-number window.

7. The method of claim 1, wherein the threshold value of step (b) is determined by combinations of ECG leads with an SNR that is not maximized and the combinations of ECG leads with an SNR above the threshold represents a maximized SNR for the combinations of ECG leads.

8. The method of claim 7, wherein step (c) includes tracking a dominant spectral peak in the ECG signals from the combination of ECG leads determined in step (b) to extract a respiratory rate of the subject from the ECG signals.

9. The method of claim 8, wherein step (c) further includes correlating a frequency of the dominant spectral peak in the ECG signals from the combination of ECG leads determined in step (b) with a respiratory rate of the subject.

10. The method of claim 2, further comprising computing a modulation of a respiration envelope signal, using the at least one of a series of root-mean-squared (RMS) amplitudes, for use in a tidal volume analysis.

11. A system for determining a respiratory rate of a subject from elctrocardiographic (ECG) signals acquired from the subject, the system comprising:
an ECG apparatus configured to acquire ECG signals from the subject through a plurality of ECG leads, wherein the plurality of ECG leads includes lead groups that are traditionally presumed to be orthogonal; and
a processor configured to:
(i) determine a combination of ECG leads from the plurality of ECG leads that provides a spectral signal-to-noise ratio (SNR) above a threshold value;
(ii) process the ECG signals from the combination of ECG leads determined in step (i) using an algorithm configured to extract a respiratory rate of the subject from the ECG signals; and
a report generator configured to provide a report of the respiratory rate of the subject.

12. The system of claim 11, wherein the processor is further configured to identify at least one of a series of root-mean-squared (RMS) amplitudes, consistent with R-wave occurrences in QRS complexes, from the ECG signals acquired for each of the plurality of ECG leads, on a beat-by-beat basis.

13. The system of claim 12, wherein the processor is further configured to determine a series of RMS amplitude ratios using a first and a second series of RMS amplitudes for the plurality of ECG lead combinations, on a beat-by-beat basis.

14. The system of claim 13, wherein the processor is further configure to determine a set of power spectra using a fast Fourier transform (FFT) of the series of RMS amplitude ratios in a pre-defined beat number window, for the plurality of ECG lead combinations.

15. The system of claim 14, wherein the processor is further configured to characterize each of the set of power spectra by an SNR defined by:
$$SNR = 10 {log}_{10} ( \frac{signal}{noise} )$$
whereby the “signal” represents the spectral peak power and the “noise” is given by a median of the power spectrum, for a frequency ranging from 0 to 0.5 cycles/beat.

16. The system of claim 15, wherein the processor is further configured to convert a frequency axis for each of the set of power spectra from a number of cycles per beat to a number of respirations per minute using a scale obtained from a heart rate estimate across a predefined beat-number window.

17. The system of claim 11, wherein the processor is further configured to determine the threshold value by identifying combinations of ECG leads with an SNR that is not maximized.

18. The system of claim 17, wherein the processor is further configured to track a dominant spectral peak in the ECG signals from the combination of ECG leads to extract a respiratory rate of the subject from the ECG signals.

19. The system of claim 18, wherein the processor is further configured to correlate a frequency of the dominant spectral peak in the ECG signals from the combination of ECG leads with a respiratory rate of the subject.

20. The system of claim 18, wherein the report generator includes a display configured to display a waveform illustrating the respiratory rate of the subject.

21. The system of claim 12, wherein the processor is further configured to compute a minute ventilation as product of respiratory rate and tidal volume.

22. A system for deriving elctrocardiographic (ECG) signals from a subject, the system comprising:
an ECG apparatus configured to acquire ECG signals from the subject through a plurality of ECG leads, wherein the plurality of ECG leads includes lead groups that are traditionally presumed to be orthogonal; and
a processor configured to:
(i) analyze combinations of ECG leads from the plurality of ECG leads to determine a spectral signal-to-noise ratio (SNR) for each combination of ECG leads;
(ii) select a combination of ECG leads that provides a desirable spectral SNR; and a report generator configured to provide a report of the ECG signals derived from the combination of ECG leads selected in by the processor as providing the desirable spectral SNR.

23. A method for determining respiratory rate of a subject from elctrocardiography (ECG) signals acquired from the subject, the method comprising the steps of:
(a) acquiring ECG signals from an ECG monitor coupled to the subject through a plurality of ECG leads, wherein the plurality of ECG leads includes lead groups that are presumed to be orthogonal;
(b) analyzing combinations of ECG leads from the plurality of ECG leads, including lead groups other than the lead groups that are presumed to be orthogonal, to determine a combination of ECG leads that provides a spectral signal-to-noise ratio (SNR) greater that other combinations of ECG leads from the plurality of ECG leads;
(c) tracking a dominant spectral peak in the ECG signals from the combination of ECG leads determined in step (b);
(d) correlating the dominate spectral peak with a respiratory rate of the subject; and
(e) generating a report indicating the respiratory rate of the subject based on step (d).

24. The method of claim 20, wherein the combination of ECG leads determined in step (b) are non-orthogonal.
